(19) United States
(12) Patent Application Publication
(10) Pub. No.: US 2012/0076858 A1
(43) Pub. Date: Mar. 29, 2012

(54) Orally Disintegrating Dosage Forms Containing Taste-Masked Active Ingredients

(75) Inventors: Karl Kolter, Limburgerhof (DE); Maximilian Angel, Schifferstadt (DE)

(73) Assignee: BASF SE, Ludwigshafen (DE)

(21) Appl. No.: 13/375,611

(22) PCT Filed: May 31, 2010
(86) PCT No.: PCT/EP10/57515

§ 371 Date: Dec. 1, 2011

(30) Foreign Application Priority Data
Jun. 4, 2009 (EP) 09161910.6

(51) Int. Cl. A61K 9/00

(52) U.S. Cl. 424/486; 424/484; 424/488

(57) ABSTRACT

Orally disintegrating dosage forms, for the purpose of masking the taste, comprise active ingredients coated with a cationic polymer are described. The coating of polymers comprises N,N-diethylaminoethyl methacrylate (DEAEMA) polymerized therein. The taste-masked active ingredients are embedded into an orally disintegrating matrix.

The present invention relates to orally disintegrating pharmaceutical dosage forms which, for the purpose of masking the taste, comprise active ingredients coated with a cationic polymer. The taste-masking coatings are applied by means of an aqueous polymer dispersion which by free-radical emulsion polymerization of a monomer mixture which comprises N,N-diethylaminoethyl methacrylate.

Rapidly orally disintegrating and/or rapidly dissolving tablets are gaining ever greater importance for the oral application of drugs. Such tablets must disintegrate within a short time, at best within 30 seconds, in the oral cavity, have a pleasant taste and must not leave behind a sandy feel. In addition, they should be easy to manufacture, with direct tableting offering considerable advantages over wet granulation, and they must have high mechanical strength so that they can withstand packaging procedures, transportation and also squeezing out from packings in an undamaged form. Chewable and suckable tablets which likewise dissolve in the mouth are also achieving greater importance.

DE-B 1090381 describes a method of coating pharmaceuticals with coating masses that are soluble in the stomach. These comprise a copolymer of 20 to 80% of at least one amino ester of (meth)acrylic acid and 80 to 20% of a monomer which forms a water-insoluble polymer as homopolymer. Specific examples of suitable polymerizable amino esters that are given are the esters of acrylic acid and (meth)acrylic acid with N,N-dimethylaminoethanol, N,N-diethylaminoethanol, N,N-dimethylaminopropanol and N-(hydroxyethyl)morpholine. Suitable comonomers that are mentioned are lower esters of acrylic acid and preferably of (meth)acrylic acid, such as ethyl acrylate, methyl, butyl and hexyl(meth)acrylates. The preparation takes place by solution polymerization in an organic solvent; a working example is not given.

DE-B 1219175 describes a method of producing veterinary medicine active ingredient preparations which are protected against the effect of rumen juices of ruminants. For this, these preparations are coated with copolymers which comprise N,N-dialkylaminoalkyl(meth)acrylamides and a comonomer polymerized therein which is selected from (meth)acrylates, acrylonitrile and vinylaromatics. Copolymers based on N,N-dialkylaminoalkyl(meth)acrylates are regarded as disadvantageous according to the teaching of this document since the ester group, compared to the amide group, rather saponifies in the basic medium.

DE-A 2135073 describes coating compositions for pharmaceuticals which comprise an aqueous polymer dispersion, where the polymer is composed to 10 to 55% by weight of monomers with a carboxyl group and/or a monoalkyl- or dialkylaminoalkyl ester group. A suitable monomer that is specified is, besides a large number of others, also diethylaminoethyl methacrylate (DEAEMA). Suitable comonomers that are mentioned are the lower esters of (meth)acrylic acid, preferably methyl methacrylate, (meth)acrylonitrile, vinylaromatics, vinyl chloride and vinyl acetate. The preparation takes place by aqueous emulsion polymerization, preferably by the emulsion feed method. Specific emulsion polymers based on DEAEMA are not disclosed.

For providing binders for pharmaceutical coatings with a low residual monomer content, DE-B 2512238 teaches the use of a powder obtained by spray-drying a polymer dispersion for producing coating solutions for these pharmaceuticals. As regards the dispersions used for the spray-drying, reference is made to DE 1090381, DE 1219175 and DE 2135073.

DE-A 2838278 describes coatings for oral administration forms for ruminants comprising
a) at least one film-forming polymer with at least one basic amino group and with a nitrogen content of from 3 to 14% which is soluble in aqueous rumen medium at a pH above 5.5 within 24 hours, and
b) at least one hydrophobic substance dispersed in the polymer which is selected from C₁₂-C₃₂-fatty acids, Al salts of these fatty acids and/or polycarboxylic acids.

To produce the coating, a solution in an organic solvent is used. A large number of nitrogen-containing homopolymers and copolymers is listed as suitable polymers, without discussing suitable processes for their preparation. Working example 29 here gives a copolymer of 40% N,N-diethylaminoethyl methacrylate, but without stating a process for its preparation.

GB 1324087 describes coating polymers for oral administration forms for ruminants which comprise
a) at least one N,N-dialkylaminoalkyl(meth)acrylate and
b) at least one ethylenically unsaturated compound which is selected from vinylaromatics and derivatives thereof, vinyl esters, esters of (meth)acrylic acid and acrylonitrile polymerized therein.

Suitable monomers a) that are disclosed are N,N-dimethylaminoethyl methacrylate (DMAEMA) and tert-butylaminoethyl methacrylate (TBAEMA). As comonomer b), in particular methyl methacrylate is considered to be unsuitable since it has a tendency to form excessively brittle coatings. Bulk polymerization, suspension polymerization, solution polymerization and emulsion polymerization are stated as suitable polymerization processes. The copolymers in the working examples were produced by solution polymerization.

DE 3426587 A1 describes a process for coating pharmaceuticals by applying a film of a liquid, film-forming coating composition which comprises a dissolved polymer with pendent tertiary ammonium salt groups. To produce these polymer solutions, it is possible to convert, inter alia, copolymers based on N,N-dialkylaminoalkyl(meth)acrylates with aqueous inorganic or organic acids into aqueous ammonium salt solutions.

DE 3049179 A1 is an application of addition to DE 2512238 and relates to the use of a powder obtained by spray-drying in accordance with the teaching of the last-mentioned document in the form of an aqueous suspension which additionally comprises a plasticizing agent for producing coatings by thermal gelation.

EP 0058765 A2 describes coating compositions for pharmaceuticals that are soluble or swellable in gastric juice which comprise, as binder, an emulsion polymer based on N,N-dialkylaminoalkyl(meth)acrylates, there being located between the amino group and the (meth)acrylate group a branched alkylene or aralkylene group having at least three carbon atoms arranged in a straight chain.

WO 2005/055986 and WO 2005/056619 describe polymers with pH-dependent swelling/dissolving behavior and their use in pharmaceuticals.

WO 00/05307 deals with the provision of coating compositions and binders for pharmaceuticals which comprise (meth)acrylate copolymers which have monomer radicals with tertiary amino groups, the aim being that simple dry or aqueous further processing be possible. For this purpose, this document teaches a process in which (a) a copolymer of C1-C4-esters of (meth)acrylic acid and (meth)acrylate monomers which have tertiary ammonium groups, (b) a plasticizer and (c) an emulsifier with an HLB value of at least 14 are blended together, and the coating composition or binder is prepared therefrom by melting, pouring, spreading or spraying, where the copolymer (a) is introduced in power form with an average particle size of from 1 to 40 μm. The processability achieved in this case is attributed to the provision of the copolymer (a) in powder form with an extremely small particle size.

WO 02/067906 relates to coating compositions and binders with improved water vapor permeability compared with those described in WO 00/05307. Here, the coating compositions and binders are prepared with a mixture which comprises (a) a copolymer of C₁-C₄-esters of (meth)acrylic acid and further (meth)acrylate monomers with functional tertiary ammonium groups in powder form with an average particle size of from 1 to 40 μm, (b) an emulsifier with an HLB value of at least 14 and (c) a C₁₂-C₁₈-monocarboxylic acid or a C₁₂-C₁₈-hydroxyl compound.

WO 2004/019918 describes coating compositions and binders which correspond in terms of their composition to those described in WO 00/05307 and WO 02/067906.

Rapidly disintegrating tablets often consist of sugars and sugar alcohols, effervescent systems, microcrystalline cellulose and other non-water-soluble fillers such as calcium hydrogenphosphate, cellulose derivatives, corn starch, or polypeptides. Furthermore, water-soluble polymers, customary disintegrants (crosslinked PVP, sodium and calcium salts of crosslinked carboxymethylcellulose, sodium salt of carboxymethyl starch, low-substituted hydroxypropylcellulose (L-HPC) and essentially inorganic water-insoluble constituents (silicas, silicates, inorganic pigments) are used. In addition, the tablets can also comprise surfactants.

WO 2003/051338 describes a directly tabletable and readily compressible auxiliary formulation which comprises mannitol and sorbitol. Firstly, an auxiliary premix is prepared by dissolving mannitol and sorbitol in water and then spray-drying (customary spray-drying and SBD methods). Additionally, mannitol can be added to this coprocessed mixture. Tablets which additionally comprise disintegrant, release agent, pigment and an active ingredient are said to disintegrate in the oral cavity within 60 seconds.

US 2002/0071864 A1 describes a tablet which disintegrates in the oral cavity within 60 seconds, and is formulated primarily from a physical mixture of spray-dried mannitol and a coarsely granular crosslinked polyvinylpyrrolidone and a limited selection of active ingredients. These tables have a breaking strength of ca. 40N and produce an unpleasant, sandy mouth feel.

According to U.S. Pat. No. 6,696,085 B2, a methacrylic acid copolymer type C should be used as disintegrant. The methacrylic acid copolymer type C is an enteric polymer which is not soluble in the acidic pH range, but is water-soluble in the pH range of 7, as is present in the oral cavity. Besides a low breaking strength (<20N), the tablets have high friability (>7%) and include a high proportion, in the regain of 15% by weight, of a coarsely granular disintegrant. Consequently, they have low mechanical strength and, on account of the high proportion of coarsely granular disintegrant, have an unpleasant, sandy mouth feel.

EP 0839526 A2 describes a pharmaceutical administration form consisting of an active ingredient, erythritol, crystalline cellulose and a disintegrant. Furthermore, mannitol is incorporated, and crosslinked polyvinylpyrrolidone is used as disintegrant, giving a physical mixture. The tablets are said to disintegrate in the oral cavity within 60 seconds.

The application JP 2004-265216 describes a tablet that disintegrates in the mouth within 60 seconds and consists of an active ingredient, a water-soluble polyvinyl alcohol-polyethylene glycol copolymer, sugar/sugar alcohol (mannitol) and disintegrant.

The matrix components based on sugar alcohols, disintegrants and insoluble polymers are generally known for pharmaceutical applications from WO 2007/071581.

WO 2009/016258 discloses the preparation of aqueous polymer dispersions of cationic polymers based on N,N-diethylaminoethyl methacrylate and their use for coating drugs.

The object of the present invention is to provide improved pharmaceutical administration forms which are suitable for use as orally disintegrating dosage forms of unpleasant tasting active ingredients.

Surprisingly, it has now been found that taste-masked active ingredients which, for masking the taste, with polymer dispersions which comprise N,N-diethylaminoethyl methacrylate (DEAEMA) in copolymerized form, are particularly suitable for such orally disintegrating dosage forms.

The coating compositions used for masking the taste are based on aqueous polymer dispersions which are obtained by free-radical emulsion polymerization of a monomer mixture M), comprising
a) N,N-diethylaminoethyl methacrylate, and
b) at least one free-radically polymerizable compound selected from esters of α,β-ethylenically unsaturated mono- and dicarboxylic acids with C₁-C₅-alkanols, in an aqueous medium at a pH of at least 7, preferably at least 8.

Monomer a)

According to the invention, N,N-diethylaminoethyl methacrylate is used as monomer a).

To prepare the aqueous polymer dispersions Pd) according to the invention, the component a) is used preferably in an amount of from 25 to 65%, particularly preferably 30 to 60%, in particular 38 to 48% by weight, specifically 43 to 47% by weight, based on the total weight of the monomers used for the polymerization.

Monomer b)

The component b) is selected from esters of α,β-ethylenically unsaturated mono- and dicarboxylic acids with C₁-C₈-alkanols.

Suitable compounds b) are methyl (meth)acrylate, methyl ethacrylate, ethyl(meth)acrylate, ethyl ethacrylate, n-propyl(meth)acrylate, isopropyl(meth)acrylate, n-butyl(meth)acrylate, sec-butyl(meth)acrylate, tert-butyl(meth)acrylate, tert-butyl ethacrylate, n-hexyl(meth)acrylate, n-heptyl(meth)acrylate, n-octyl(meth)acrylate, 1,1,3,3-tetramethylbutyl(meth)acrylate and ethylhexyl(meth)acrylate.

As component b), particular preference is given to using methyl methacrylate or a monomer mixture comprising methyl methacrylate.

To prepare the aqueous polymer dispersions according to the invention, the component b) is used preferably in an amount of from 35 to 75% by weight, particularly preferably 40 to 70% by weight, in particular 52 to 62% by weight, specifically 53 to 57% by weight, based on the total weight of the monomers used for the polymerization.

The monomer mixtures M) used for the preparation of the polymer dispersions can additionally comprise at least one further monomer c). The additional monomers c) are preferably selected from esters of α,β-ethylenically unsaturated mono- and dicarboxylic acids with C₉-C₃₀-alkanols and C₂-C₃₀-alkanediols, amides of α,β-ethylenically unsaturated mono- and dicarboxylic acids with C₂-C₃₀-aminoalcohols which have a primary or secondary amino group, primary amides of α,β-ethylenically unsaturated monocarboxylic acids and N-alkyl and N,N-dialkyl derivatives thereof, N-vinyllactams, open-chain N-vinylamide compounds, esters of vinyl alcohol and allyl alcohol with C₁-C₃₀-monocarboxylic acids, vinyl ethers, vinylaromatics, vinyl halides, vinylidene halides, C₂-C₈-monoolefins, unsaturated nitriles, nonaromatic hydrocarbons with at least two conjugated double bonds and mixtures thereof.

Suitable additional monomers c) are esters of α,β-ethylenically unsaturated mono- and dicarboxylic acids with C₉-C₃₀-alkanols, such as n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arachinyl(meth)acrylate, behenyl(meth)acrylate, lignoceryl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable additional monomers c) are also esters of α,β-ethylenically unsaturated mono- and dicarboxylic acids with C₂-C₃₀-alkanediols, such as 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate, 3-hydroxy-2-ethylhexyl methacrylate etc.

Suitable additional monomers c) are also primary amides of α,β-ethylenically unsaturated monocarboxylic acids and the N-alkyl and N,N-dialkyl derivatives thereof, such as acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachinyl(meth)acrylamide, N-behenyl(meth)acrylamide, N-lignoceryl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide, N,N-dimethyl(meth)acrylamide, N,N-diethyl(meth)acrylamide, morpholinyl(meth)acrylamide.

Suitable additional monomers c) are also N-vinyllactams and derivatives thereof which can have e.g. one or more C₁-C₆-alkyl substituents, such as methyl, ethyl, n-propyl, isopropyl, n-butyl, sec-butyl, tert-butyl etc. These include e.g. N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preference is given to using N-vinylpyrrolidone and N-vinylcaprolactam.

Open-chain N-vinylamide compounds suitable as monomers c) are, for example,

N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Suitable additional monomers c) are also vinyl acetate, vinyl propionate, vinyl butyrate and mixtures thereof.

Suitable additional monomers c) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The aforementioned additional monomers c) can be used individually or in the form of any desired mixtures.

To prepare the aqueous polymer dispersions according to the invention, the component c) is preferably used in an amount of from 0 to 80% by weight, based on the total weight of the monomers used for the polymerization. A specific embodiment relates to polymer dispersions Pd) which comprise no additional monomer c) polymerized therein. If present, the component c) is used preferably in an amount of from 0.1 to 70% by weight, particularly preferably 1 to 60% by weight, in particular 5 to 50% by weight, based on the total weight of the monomers used for the polymerization.

Preferably, no monomer c) is used.

Monomer d)

The monomer mixtures M) used to prepare the polymer dispersions can comprise, in addition to compound a), at least one further compound d) different therefrom and having a free-radically polymerizable α,β-ethylenically unsaturated double bond and at least one cationogenic and/or cationic group per molecule polymerized therein.

Preferably, the cationogenic and cationic groups of component d) are nitrogen-containing groups, such as primary, secondary and tertiary amino groups, and also quaternary ammonium groups. Preferably, the nitrogen-containing groups are tertiary amino groups or quaternary ammonium groups. Charged cationic groups can be produced from the amine nitrogens either by protonation, e.g. with monobasic or polybasic carboxylic acids, such as lactic acid or tartaric acid, or mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

Suitable compounds d) are e.g. the esters, different from DEAEMA, of α,β-ethylenically unsaturated mono- and dicarboxylic acids with amino alcohols. Preferred amino alcohols are C₂-C₁₂-amino alcohols which are C₁-C₈-mono- or dialkylated on the amine nitrogen. Suitable acid components of these esters are e.g. acrylic acid, methacrylic acid, fumaric acid, maleic acid, itaconic acid, crotonic acid, maleic anhydride, monobutyl maleate and mixtures thereof. As acid component of these esters, preference is given to using acrylic acid, methacrylic acid and mixtures thereof.

Suitable additional compounds d) are N,N-dimethylaminomethyl(meth)acrylate, N,N-dimethylaminoethyl(meth)acrylate, N,N-diethylaminoethyl acrylate, N,N-dimethylaminopropyl(meth)acrylate, N,N-diethylaminopropyl(meth)acrylate and N,N-dimethylaminocyclohexyl(meth)acrylate.

Suitable monomers d) are also the amides of the aforementioned α,β-ethylenically unsaturated mono- and dicarboxylic acids with diamines which have at least one primary or secondary amino group. Preference is given to diamines which have one tertiary and one primary or secondary amino group.

These include N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexygacrylamide, N-[4-(dimethylamino)cyclohexyl]methacrylamide etc.

Suitable monomers d) are also N,N-diallylamines and N,N-diallyl-N-alkylamines and their acid addition salts and quaternization products. Alkyl here is preferably C₁-C₂₄-alkyl. Preference is given to N,N-diallyl-N-methylamine and N,N-diallyl-N,N-dimethylammonium compounds, such as e.g. the chlorides and bromides.

Suitable monomers d) are also vinyl- and allyl-substituted nitrogen heterocycles, such as N-vinylimidazole, N-vinyl-2-methylimidazole, vinyl- and allyl-substituted heteroaromatic compounds, such as 2- and 4-vinylpyridine, 2- and 4-allylpyridine, and the salts thereof.

To prepare the aqueous polymer dispersions Pd) according to the invention, the component d), if present, is preferably used in an amount such that the sum of the amount of component a) and of component d) is in a range from 25 to 65% by weight, particularly preferably 30 to 60% by weight, based on the total weight of the monomers used for the polymerization.

To prepare the aqueous polymer dispersions Pd) according to the invention, the component d) is preferably used in an amount of from 0 to 50% by weight, based on the total weight of the monomers used for the polymerization.

As already explained, it has surprisingly been found that the polymer dispersions Pd) based on DEAEMA (component a)) according to the invention and used according to the invention have a particularly good profile of properties. This profile of properties can generally be achieved without using further monomers having cationogenic/cationic groups. A specific embodiment therefore relates to polymer dispersions Pd) which comprise no additional monomer d) polymerized therein.

If present, the component d) is used preferably in an amount of from 0.1 to 40% by weight, particularly preferably 1 to 30% by weight, in particular 2 to 25% by weight, based on the total weight of the monomers used for the polymerization.

In one particularly preferred embodiment of the process according to the invention, a monomer mixture M) is used which consists of
43 to 47% by weight, based on the total weight of the monomers used for the polymerization, of N,N-diethylaminoethyl methacrylate a), and
53 to 57% by weight, based on the total weight of the monomers used for the polymerization, of at least one compound b), in particular methyl methacrylate.

As regards the preparation of the polymers by free-radical emulsion polymerization, reference is hereby expressly made to the disclosure in WO 2009/016258, in which the preparation and preferred embodiments are described in detail.

The polymers present in the dispersions according to the invention preferably have an average molecular weight M_{w}, determined by means of gel permeation chromatography, in the range from 30 000 to 500 000, particularly preferably 60 000 to 140 000, in particular 80 000 to 120 000 g/mol.

The polymers present in the dispersions Pd) according to the invention preferably have a K value (determined in accordance with Fikentscher on a 1% strength solution in N-methylpyrrolidone (NMP)) in the range from 40 to 60.

The glass transition temperature TG (determined by means of DSC) is preferably in a range from 40 to 70° C., particularly preferably 52 to 62° C.

The polymers present in the dispersions according to the invention are essentially random copolymers.

The average particle diameter of the polymer particles present in the polymer dispersion (determined by means of analytical ultracentrifuge) is preferably in a range from 70 to 200 nm, particularly preferably from 80 to 150 nm, especially from 90 to 120 nm. The particle size distribution is preferably essentially unimodal.

The LT value of the dispersions according to the invention, determined on a 0.01% strength dispersion in water (2.5 cm cuvette, white light) is preferably at least 70%, particularly preferably at least 80%. The determination of the light transparency is described e.g. in Dieter Distler, Wassrige Polymerdispersionen [Aqueous polymer dispersions], Wiley-VCH (1999), p. 40.

The solids content of the dispersions according to the invention is preferably 10 to 50% by weight, particularly preferably 20 to 40% by weight. In the case of a purification of the dispersion by means of ultrafiltration, the dispersions according to the invention preferably have solids contents which are within these ranges before and after the ultrafiltration. It is of course likewise possible to subject a dilute polymer dispersion to concentration by ultrafiltration.

The dispersions used according to the invention for masking the taste have extremely low viscosities of preferably less than 50 mPas, particularly preferably less than 25 mPas and in particular less than 10 mPas (values determined by means of Brookfield viscometer at 20° C. and 100 s⁻¹) e.g. even in the case of a solids content of 30% by weight. Such low viscosities are of particular importance for many applications.

The charge of the polymers present in the dispersions according to the invention is dependent on the pH of the dispersion. The isoelectric point is preferably in a pH range from about 7.5 to 8.5. The finished dispersion preferably has a pH in the range from 8 to 10, particularly preferably from 8.5 to 9.5 (at a solids content of 30% by weight). It is advantageous that the selected pH of the finished dispersion is higher (more strongly alkaline) than its isoelectric point, provided dissolution or swelling of the polymer particles present in the dispersion is not desired. The dispersions according to the invention are therefore preferably basic dispersions.

The polymer dispersions according to the invention are characterized by their pH-dependent solubility. An adjustment of the pH range in which the dispersion dissolves upon acidification is possible, e.g. through the amount of N,N-diethylaminoethyl methacrylate (monomer a) polymerized therein and also optionally the use of further monomers with cationogenic/cationic groups (monomer d). Preferably, the polymers present in the polymer dispersions Pd) according to the invention dissolve at a pH of at most 6.8, particularly preferably at a pH of at most 5.5.

According to one preferred embodiment, polymer dispersions are used which comprise a polymer which comprises
43 to 47% by weight, based on the total weight of the monomers used for the polymerization, of N,N-diethylaminoethyl methacrylate a), and
53 to 57% by weight, based on the total weight of the monomers used for the polymerization, of at least one compound b)
   as the only monomers polymerized therein.

The coating compositions for pharmaceutical administration forms used according to the invention can comprise at least one further pharmaceutically acceptable auxiliary. Pharmaceutically acceptable auxiliaries are those which are known for use in the field of pharmacy, food technology and related fields, in particular those listed in the relevant pharmacopoeia (e.g. Ph. Eur., USP, JP), and also other auxiliaries, whose properties do not preclude a physiological application.

Suitable auxiliaries may be: aroma substances, taste-improving substances, sweetening agents (sugars, sugar alcohols, sweeteners such as e.g. aspartame, saccharine-Na, sodium cyclamate), glidants, wetting agents, release agents, plasticizers, antiadhesives, stabilizers, pore formers, neutralizing agents, gloss agents, dyes, pigments, disinfectants or preservatives, thickeners, etc. Such substances are described e.g. in Fiedler, H. P. Lexikon der Hilfsstoffe fur Pharmazie, Kosmetik and angrenzende Gebiete [Lexicon of auxiliaries for pharmacy, cosmetics and related fields], 4th edition, Aulendorf: ECV-Editio-Cantor-Verlag, 1996.

Customary amounts of the auxiliaries are in a range of in each case from 0 to 50% by weight, preferably 0 to 20% by weight, in particular 0.01 to 10% by weight, based on the total weight of the coating composition.

The coating compositions can be produced e.g. by intimate mixing of a polymer dispersion according to the invention or of a polymer obtainable therefrom by a drying process with at least one auxiliary.

The coating composition according to the invention can be used e.g. in powder form, as melt or in aqueous emulsion by granulation, pouring, spreading or by means of spray application. Preference is given to application as polymer dispersion, specifically as primary dispersion. The coating compositions according to the invention can additionally comprise at least one further polymer component. In this connection, it is possible to use mixtures of at least two dispersions, at least one dispersion and at least one solution, at least one dispersion and at least one powder, at least two powders, etc.

The formulation according to the invention is suitable for the administration of in principle any desired pharmaceutical active ingredients which can preferably be administered in isolated or protected form, such as antidepressants, beta receptor blockers, antidiabetics, analgesics, antiphlogistics, antirheumatics, antihypotonics, antihypertonics, psychopharmacueticals, tranquilizers, antiemetics, muscle relaxants, glucocorticoids, agents for treating Colitis ulcerosa or Crohn's disease, antiallergics, antibiotics, antiepileptics, anticoagulants, antimycotics, antitussives, arteriosclerosis drugs, diuretics, enzymes, enzyme inhibitors, gout remedies, hormones and inhibitors thereof, cardiac glycosides, immunotherapeutics and cytokines, laxatives, antilipemics, gastrointestinal therapeutics, migraine remedies, preparations of minerals, otologics, Parkinson's drugs, thyroid therapeutics, spasmolytics, platelet aggregation inhibitors, vitamins, cytostatics and metastasis inhibitors, phytopharmaceuticals, chemotherapy drugs, nutraceuticals, vitamins, carotenoids and amino acids.

Examples of suitable active ingredients are: acarbose, nonsteroidal antirheumatics, cardiac glycosides, acetylsalicylic acid, virustatic agents, aclarubicin, aciclovir, cisplatin, actinomycin, α- αnd β-sympatomimetics, allopurinol, alosetron, alprostadil, prostaglandins, amantadine, ambroxol, amlodipine, methotrexate, 5-aminosalicylic acid, amitriptyline, amlodipine, amoxicillin, anastrozole, atenolol, atorvastatin, azathioprin, balsalazide, beclomethasone, betahistine, bezafibrate, bicalutamide, diazepam and diazepam derivatives, budesonid, bufexamac, buprenorphine, methadone, calcium salts, potassium salts, magnesium salts, candesartan, carbamazepine, captopril, cefalosporins, celetoxib, cetirizine, chenodeoxycholic acid, ursodeoxycholic acid, theophylline and theophylline derivatives, trypsin, cimetidine, clarithromycin, clavulanic acid, clindamycin, clobutinol, clonidine, cotrimoxazole, codeine, caffeine, vitamin D and derivatives of vitamin D, colestyramine, cromoglycic acid, coumarin and coumarin derivatives, cysteine, cytarabine, cyclophosphamide, cyclosporin, cyproterone, cytarabine, dapiprazole, desogestrel, desonid, dihydralazine, diltiazem, ergot alkaloids, dimenhydrinate, dimethylsulfoxide, dimeticone, dipyridamole, domperidone and domperidone derivatives, donepzil, dopamine, doxazosin, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinanes, calcium antagonists, irinotecan, modafinil, orlistat, peptide antibiotics, phenytoin, riluzole, risedronate, sildenafil, topiramat, macrolid antibiotics, esomeprazole, estrogen and estrogen derivatives, gestagen and gestagen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, flunarizine, fluorouracil, fluoxetine, flurbiprofen, ibuprofen, flutamide, fluvastatin, follitropin, formoterol, fosfomycin, furosemide, fusidic acid, galantamine, gallopamil, ganciclovir, gemfibrozil, gentamicin, ginkgo, St. John's wort, glibenclamide, urea derivatives as oral antidiabetics, glucagon, glucosamine and glucosamine derivatives, glutathione, glycerol and glycerol derivatives, hypothalamus hormones, goserelin, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramin, insulin, interferons, iodine and iodine derivatives, isoconazole, isoprenalin, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquin, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastin, moexipril, morphine and morphine derivatives; evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamin sulfone, noscapine, nystatin, ofloxacin, olanzapine, olsalazine, omeprazole, omoconazole, ondansetron, orlistat, oseltamivir, oxaceprol, oxacillin, oxiconazole, oxymetazolin, pantoprazol, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirol, rosiglitazone, roxatidine, roxithromycin, ruscogenin, rutoside and rutoside derivatives, sabadilla, salbutamol, salmeterol, scopolamine, selegiline, sertaconazole, sertindole, sertraline, silicates, simvastatin, sitosterol, sotalol, spaglumic acid, sparfloxacin, spectinomycin, spiramycin, spirapril, spironolactone, stavudine, streptomycin, sucralfate, sufentanil, sulbactam, sulfonamides, sulfasalazine, sulpiride, sultamicillin, sultiame, sumatriptan, suxamethonium chloride, tacrine, tacrolimus, taliolol, tamoxifen, taurolidine, tazarotene, tegaserod, temazepam, teniposide, tenoxicam, terazosin, terbinafine, terbutaline, terfenadine, terlipressin, tertatolol, tetracyclines, tetryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antiestrogens, tramadol, tramazoline, trandolapril, tranylcypromine, trapidil, trazodon, triamcinolon and triamcinolon derivatives, triamterene, trifluperidol, trifluridine, trimethoprim, trimipramine, tripelennamine, triprolidine, trifosfamide, tromantadine, trometamol, tropalpine, troxerutin, tulobuterol, tyramine, tyrothricin, urapidil, ursodeoxycholic acid, chenodeoxycholic acid, valaciclovir, valdecoxib, valproic acid, vancomycin, vecuronium chloride, venlafaxine, verapamil, vidarabine, vigabatrin, viloxazine, vinblastine, vincamine, vincristine, vindesine, vinorelbine, vinpocetine, viquidil, warfarin, xantinol nicotinate, xipamide, zafirlukast, zalcitabine, zanamivir, zidovudine, zolmitriptan, zolpidem, zopiclone, zotepine and the like.

If desired, the active ingredients can also be used in the form of their pharmaceutically acceptable salts or derivatives, and, in the case of chiral active ingredients, it is possible to use either optically active isomers or racemates or diastereoisomer mixtures. If desired, the compositions according to the invention can also comprise two or more pharmaceutical active ingredients.

According to the invention, the active ingredients coated to mask the taste can be used in the form of extrudates, mini tablets, capsules, soft capsules, granulates, pellets, micropellets, microcapsules or crystals. The particle size of the coated active ingredient forms is <1000 μm, preferably <500 μm, particularly preferably 25 to 250 μm, in particular 50 to 150 μm.

To prepare the orally disintegrating dosage forms, the coated granulates, pellets, micropellets, microcapsules, crystals can be mixed with suitable auxiliaries and compressed to give tablets which disintegrate in the aqueous medium of the oral cavity and release the coated fine molded articles again. Of particular importance here are so-called oral dispersibles, i.e. tablets which disintegrate in the mouth within a short time and release the taste-masked small molded articles.

Active ingredient classes and substances which can often cause an unpleasant bitter taste and which can advantageously be formulated according to the invention are e.g.:

Analgesics and antirheumatics, such as paracetamol, diclofenac, aceclofenac, ibuprofen, ketoprofen, flubiprofen, acetylsalicylic acid, levacetylmethadol and oxycodone;

psychopharmaceuticals, such as promethazine, donepezil, modafinil, nefazodone, reboxetine, sertindole and sertraline;

antibiotics, such as erythromycin, roxithromycin, clarithromycin, grepafloxacin, ciprofloxacin, levofloxacin, sparfloxacin, trovafloxacin and nevirapine;

betablockers, such as propranolol, metoprolol, bisoprolol and nebivolol;

antidiabetics, such as metformin, miglitol and repaglinide;

H₁ antihistamines, such as diphenhydramine, fexofenadine and mizolastine;

H₂ antihistamines, such as cimetidine, famotidine, roxatidine, nizatidine, ticlopidine, cetirizine and ranitidine;

vitamins such as thiamine nitrate and also quinidine sulfate, amyloprilose HCl, pseudoephedrine HCl, sildenafil, topiramate, granisetron, rebamipide, quinine HCl, etc.

The exceptional taste masking results from the insolubility of the polymers according to the invention at pH values greater than 6 and the rapid solubility at pH values below 6. This means in the saliva (pH: 7.2), accordingly coated forms are stable for a very long time and there is no contact between the bitter drug and the oral mucosa, whereas in the stomach at pH values of 1 to 5, very rapid release of the active ingredient takes places. The dissolution is so rapid that there is virtually no difference in the onset of effect compared with an uncoated form. As a rule, film coatings of a polymer according to the invention dissolve in gastric juice within 5 min, whereas they are stable for more than 2 hours in phosphate buffer pH 7.2. Surprisingly, the film coatings also dissolve relatively rapidly in media with pH values of 4.5, meaning that the administration forms produced therefrom also develop a rapid effect in the case of antacid patients or patients, who are treated with antacids.

The coating compositions according to the invention have low water vapor and oxygen permeability and consequently permit the formulation and stabilization of particularly water-vapor-sensitive or oxygen-sensitive drugs, such as e.g. acetylsalicylic acid, enalapril, cortisone acetate, omeprazole, carotenoids. Here, the coating has a protective character.

Moreover, the coating compositions according to the invention can be used for separating incompatible active ingredients or auxiliaries in administration forms by surrounding one or more constituents and thus avoiding mutual contact.

The unexpectedly very good application properties of the film coatings according to the invention are made possible by an excellent homogeneous filming of the polymer dispersion, low stickiness of the films and good flexibility and/or extensibility of the coatings, meaning that even if the tablet or pellet core swells, the film coating does not tear. Here, especially the combination of high flexibility with extremely low stickiness is surprising since normally polymers are either hard, i.e. not very flexible and not sticky, or are soft, i.e. flexible but sticky.

Suitable matrices for the orally disintegrating dosage forms are in principle all auxiliaries or auxiliary mixtures known for this purpose.

Suitable auxiliaries or auxiliary mixtures for the matrix are, in particular, those based on sugars or sugar alcohols. Suitable sugars or sugar alcohols are mannose, trehalose, mannitol, erythritol, isomalt, maltitol, lactitol, xylitol, sorbitol.

Of suitability in this connection are also mixtures of gelatin and sugar alcohols as bases for a matrix. The aqueous mixtures can be mixed with the taste-masked active ingredient molded articles, introduced into molds and solidified to give dosage forms. The solidification can preferably take place by lyophilization.

For the matrix, it is also possible to use co-processed sugar (alcohols). The co-processing can take place by spraying or spinning a solution of the components.

It is also possible to prepare effervescent tablets. For this, a matrix material is used which, besides the sugar (alcohol) components, comprises effervescent mixtures. Suitable effervescent mixtures consist e.g. of citric acid and sodium bicarbonate.

As matrix component, preference is given to a co-processed mixture of
a) 60-98% by weight of at least one sugar or sugar alcohol or mixtures thereof,
b) 1-25% by weight of a disintegrant,
c) 1-15% by weight of water-insoluble polymers,
d) 0-15% by weight of water-soluble polymers, and
e) 0-15% by weight of further auxiliaries,
   where the sum of components a) to e) is 100% by weight.

The preparations comprise, as component a), 60 to 98% by weight, preferably 70 to 95% by weight, particularly preferably 75 to 93% by weight, of a sugar, sugar alcohol or mixtures thereof. Suitable sugars or sugar alcohols are trehalose, mannitol, erythritol, isomalt, maltitol, lactitol, xlitol, sorbitol. The sugar or sugar alcohol components are preferably finely divided, having average particle sizes from 5 to 100 μm. If desired, the particle sizes can be adjusted by grinding.

Preference is given to using mannitol, erythritol or mixtures thereof.

As component b), disintegrants are used in amounts of from 1 to 25% by weight, preferably 2 to 15% by weight, particularly preferably 3 to 10% by weight. Such disintegrants are water-insoluble, but not film-forming. A suitable disintegrant is crosslinked polyvinylpyrrolidone (crospovidone), croscarmellose, a crosslinked carboxymethylcellulose, where, according to the invention, croscarmellose is also intended to mean its sodium and calcium salts. Sodium carboxymethyl starch is also suitable. Likewise of suitability is L-hydroxypropylcellulose, preferably with 5 to 16% hydroxypropoxy groups, as described in USP/NF 2005. Preference is given to using crospovidone.

As component c), water-insoluble polymers are used in amounts of from 1 to 15% by weight, preferably 1 to 10% by weight. Preference is given to polymers which are insoluble in the pH range from 1 to 14, i.e. have a pH-independent water insolubility at any pH. Furthermore, polymers which are water-insoluble at any pH in the pH range from 6 to 14 are also suitable.

The polymers should be film-forming polymers. In this connection, film-forming means that the polymers have, in aqueous dispersion, a minimum film-forming temperature of from −20 to +150° C., preferably 0 to 100° C.

Suitable polymers are polyvinyl acetate, ethylcellulose, methyl methacrylate-ethyl acrylate copolymers, ethyl acrylate-methyl methacrylate-trimethylammonium ethyl methacrylate terpolymers. Butyl methacrylate-methyl methacrylate-dimethylaminoethyl methacrylate terpolymers.

The acrylate-methacrylate copolymers are described in more detail in the European pharmacopoeia as Polyacrylate Dispersion 30%, in the USP as Ammonio Methacrylate Copolymer and in JPE as Aminoalkyl-Methacrylate Copolymer E.

Polyvinyl acetate is used as preferred component c). This can be used as aqueous dispersion with solids contents of from 10 to 45% by weight. Moreover, preference is given to polyvinyl acetate with a molecular weight between 100 000 and 1 000 000 daltons, particularly preferably between 200 000 and 800 000 daltons.

Furthermore, the formulations can comprise water-soluble polymers in amounts of from 0 to 15% by weight as components d). Suitable water-soluble polymers are, for example, polyvinylpyrrolidones, vinylpyrrolidone-vinyl acetate copolymers, polyvinyl alcohols, polyvinyl alcohol-polyethylene glycol graft copolymers, polyethylene glycols, ethylene glycol-propylene glycol block copolymers, hydroxypropylmethylcellulose, hydroxypropylcellulose, hydroxyethylcellulose, carragenans, pectins, xanthans, alginates.

If desired, by adding pharmaceutically customary auxiliaries (components e)) in amounts of from 0 to 15% by weight, for example such as acidifying agents, buffer substances, sweeteners, aromas, taste enhancers and dyes, it is possible to further improve the taste and appearance of the tablets obtained from the formulations.

The following substances are particularly suitable here:

Suitable acidifying agents are, for example, citric acid, tartaric acid, ascorbic acid and sodium dihydrogenphosphate.

Suitable sweeteners are, for example, cyclamate, saccharine-Na, aspartame and neohesperidin.

Suitable aromas are, for example, fruit aromas, vanilla aroma, cocoa aroma, glutamate.

Suitable dyes are: riboflavin, curcumin, betacarotene, water-soluble dyes as are used for coloring foods, and also finely divided colored lakes.

By adding thickeners such as high molecular weight polysaccharides, the mouth feel can be additionally improved by increasing the softness and the feel of volume.

Furthermore, surfactants can also be added as components e). Suitable surfactants are, for example, sodium lauryl sulfate, dioctyl sulfosuccinate, alkoxylated sorbitan esters such as Polysorbat 80, polyalkoxylated derivatives of castor oil or hydrogenated castor oil, for example Cremophor® RH 40, alkoxylated fatty acids, alkoxylated hydroxy fatty acids, alkoxylated fatty alcohols, alkali metal salts of fatty acids and lecithins. Sodium stearyl fumarate is also suitable.

Furthermore, to further improve the disintegration, it is also possible to add finely divided pigments because they increase the internal interfaces and, as a result, water is able to penetrate more rapidly into the tablet. These pigments, such as iron oxides, titanium dioxide, colloidal or precipitated silica, calcium carbonates, calcium phosphates, must naturally be very finely divided, otherwise a grainy taste will again arise.

The mixture is co-processed, for example by co-spraying, granulation or agglomeration.

The mixture of components a) to e) is preferably used in the form of agglomerates.

The formulation base of pharmaceutical compositions according to the invention preferably comprises pharmaceutically acceptable auxiliaries. Pharmaceutically acceptable auxiliaries are those which are known for use in the field of pharmacy, food technology and related fields, in particular those listed in the relevant pharmacopoeia (e.g. DAB, Ph. Eur., BP, USP, JP), and also other auxiliaries, the properties of which do not preclude a physiological application.

The matrix formulations can comprise further suitable auxiliaries. Suitable auxiliaries may be: glidants, wetting agents, emulsifying and suspending agents, preservatives, antioxidants, antiirritants, chelating agents, emulsion stabilizers, film formers, gel formers, odor masking agents, resins, hydrocolloids, solvents, solubility promoters, neutralizers, permeation accelerators, pigments, dyes, stabilizers, disintegrants, drying agents, opacifiers, thickeners, waxes, plasticizers, aromas, sweeteners, auxiliaries for lowering the permeation etc. An embodiment in this respect is based on expert knowledge, as described for example in Fiedler, H. P. Lexikon der Hilfsstoffe fur Pharmazie, Kosmetik and angrenzende Gebiete [Lexicon of auxiliaries for pharmacy, cosmetics and relates fields], 4th edition, Aulendorf: ECV-Editio-Cantor-Verlag, 1996.

Particularly suitable plasticizers are e.g.: triethyl citrate, tributyl citrate, triacetin, acetyl triethyl citrate, Labrasol, glycofurol, polypropylene glycol 400.

The permeability of the film coatings can be further reduced by incorporating inorganic solids (pigments such as, e.g. talc, kaolin, titanium dioxide) or lipophilic organic solids such as fats, waxes, glycerides, fatty acids such as e.g. stearic acid, fatty alcohols such as e.g. stearyl alcohol.

The layer thicknesses of the taste-masking coatings are between 1 μm and 100 μm, preferably between 2 and 60 μm and particularly preferably between 5 and 40 μmm. In the case of incompatibilities between active ingredient and coating, a so-called subcoating between core and taste-masking coating can be applied. This prevents direct contact between the active ingredient and the taste-masking coating. Such incompatibilities can originate e.g. from acidic active ingredients which enter into a salt formation with the basic polymer within the coating, or from active ingredients which permeate into the coating and act as plasticizer. Suitable polymers for a subcoating are water-soluble polymers such as e.g. polyvinyl alcohol, polyvinyl alcohol-polyethylene glycol graft copolymers (Kollicoat IR), polyethylene glycols, polyvinylpyrrolidones, vinylpyrrolidone-vinyl acetate copolymers, gelatin, maltodextrins, poloxamers, hydroxypropylcellulose, hydroxypropylmethylcellulose, methylcellulose. This subcoating can also comprise the customary auxiliaries for film coatings such as plasticizers, pigments, stabilizers, surfactants and also, in a small amount, also water-insoluble polymers.

The layer thicknesses of the subcoatings are between 0.5 μm and 50 μm, preferably between 1 and 30 μm and particularly preferably between 2 and 20 μm.

The invention is illustrated by reference to the following, nonlimiting examples.

### EXAMPLE 1

### Preparation of a Polymer Dispersion

Initial Charge:
481.75 kg demineralized water
5.59 kg C₁₆-/C₁₈-alkyl polyglycol ether with ca. 20 ethylene oxide units, pharmaceutical grade, 10% strength aqueous solution
4.58 kg sodium lauryl sulfate GMP, 15% strength aqueous solution

Addition 1:
14.60 kg demineralized water
0.38 kg sodium persulfate

Feed 1:
248.52 kg demineralized water
86.43 kg C₁₆-/C₁₈-alkyl polyglycol ether with ca. 20 ethylene oxide units, pharmaceutical grade, 10% strength aqueous solution
71.38 kg sodium lauryl sulfate GMP, 15% strength aqueous solution

Feed 2:
172.00 kg diethylaminoethyl methacrylate
258.00 kg methyl methacrylate

Feed 3:
153.09 kg demineralized water
3.92 kg sodium persulfate

By means of suitable technical measures (rinsing with acetone and/or blow-drying), it is ensured that the feed tank (feed 2) is largely water-free. The addition 1 and the feed 3 are freshly prepared (dissolution of solid sodium persulfate in demineralized water), directly, i.e. one hour, before the start of the polymerization. The dynamic mixer (Megatron MT 3-61, Kinematica AG) is filled with water before the start of the experiment.

The polymerization reactor (tank volume ca. 2050 l) and all of the lines coming into contact with the polymer dispersion are rinsed with a 3% strength aqueous solution of sodium hydroxide before the start of the experiment. The polymerization reactor is then filled with the initial charge. The initial charge is evacuated before the start of the reaction, gassed once with 5 bar of nitrogen, evacuated again and brought to atmospheric pressure with nitrogen. The initial charge is then heated with stirring to a reaction temperature of 75° C. Upon reaching an internal temperature of 70° C., addition 1 is added over the course of two minutes.

Feeds 1 and 2 are metered into the reactor via the dynamic mixer (rotational speed setting 5000 rpm), feed 3 is metered into the reactor via a static mixer which is located in the line section between dynamic mixer and polymerization reactor.

Feed 1 is started directly before feeds 2 and 3. The addition of feed 1 take place over the course of 1.75 hours, of feed 2 over the course of 1.50 hours, of feed 3 over the course of 3.75 hours.

When feed 3 is complete, the mixture is left to after-polymerize for a further 2 hours with stirring at 75° C. The reaction mixture is then cooled to room temperature and the solids content and also pH are determined. The pH during the polymerization (more specifically: during the addition of the monomers) was always higher than 8.0 for example 1 and examples 2 to 6 below. The K values were determined for all of the examples as 1% strength in NMP.

| | | Measurement value or |
|---|---|---|
| Parameters of the dispersion | Unit | assessment |
| Solids content | (% by wt.) | 32.7 |
| pH | | 9.0 |

The dispersion is then ultrafiltered and the following parameters determined:

| | | Measurement value or |
|---|---|---|
| Parameters of the dispersion | Unit | assessment |
| Solids content | (% by wt.) | 30.5 |
| Viscosity | (mPas) | 6 |
| pH | | 9.2 |
| LT value | (%) | 86 |
| K value | | 50 |
| Average particle size | (nm) | 100 |
| (determined by means of AUC) | | |
| Methanol | (ppm) | 20 |
| Methacrylic acid | (ppm) | 60 |
| N,N-Diethylethanolamine | (ppm) | 280 |
| Storage stability (18 months) | | excellent, no sediment |

AUC = Analytic ultracentrifuge

### EXAMPLE 2

The procedure is as in example 1 but using only 2.2 kg of sodium persulfate in feed 3, and additionally 0.4 kg of ethylhexyl thioglycolate in feed 2.

| | | Measurement value or |
|---|---|---|
| Parameters of the dispersion | Unit | assessment |
| Solids content | (% by wt.) | 31.9 |
| pH | | 9.1 |

The dispersion is then ultrafiltered and the following parameters determined:

| | | Measurement value or |
|---|---|---|
| Parameters of the dispersion | Unit | assessment |
| Solids content | (% by wt.) | 30 |
| Viscosity | (mPas) | 6 |
| pH | | 9.2 |
| LT value | (%) | 87 |
| K value | | 52 |
| Average particle size | (nm) | 105 |
| (determined by means of AUC) | | |
| Methanol | (ppm) | 18 |
| Methacrylic acid | (ppm) | 48 |
| N,N-Diethylethanolamine | (ppm) | 240 |
| Storage stability (after | | excellent, minimal sediment |
| 18 months) | | |

### EXAMPLE 3

The procedure is as in example 1, but using 193.5 kg of diethylaminoethyl methacrylate and 236.5 kg of methyl methacrylate in feed 2.

| | | Measurement value or |
|---|---|---|
| Parameters of the dispersion | Unit | assessment |
| Solids content | (% by wt.) | 30.3 |
| pH | | 9.0 |

The dispersion is then ultrafiltered and the following parameters determined:

| | | Measurement value or |
|---|---|---|
| Parameters of the dispersion | Unit | assessment |
| Solids content | (% by wt.) | 30 |
| Viscosity | (mPas) | 5 |
| pH | | 9.2 |
| LT value | (%) | 89 |
| K value | | 50 |
| Average particle size | (nm) | 110 |
| (determined by means of AUC) | | |
| Methanol | (ppm) | 22 |
| Methacrylic acid | (ppm) | 65 |
| N,N-Diethylethanolamine | (ppm) | 210 |
| Storage stability (after | | excellent, minimal sediment |
| 18 months) | | |

### EXAMPLE 4

Initial Charge:
378.24 kg demineralized water
5.59 kg C₁₆-/C₁₈-alkyl polyglycol ether with ca. 20 ethylene oxide units, pharmaceutical grade, 10% strength aqueous solution
4.58 kg sodium lauryl sulfate GMP, 15% strength aqueous solution

Addition 1:
14.60 kg demineralized water
0.38 kg sodium persulfate

Feed 1:
352.03 kg demineralized water
86.43 kg C₁₆-/C₁₈-alkyl polyglycol ether with ca. 20 ethylene oxide units, pharmaceutical grade, 10% strength aqueous solution
71.38 kg sodium lauryl sulfate GMP, 15% strength aqueous solution

Feed 2:
172.00 kg diethylaminoethyl methacrylate
258.00 kg methyl methacrylate

Feed 3:
153.09 kg demineralized water
3.92 kg sodium persulfate

The polymerization reactor (tank volume ca. 2050 l) and all of the lines coming into contact with the polymer dispersion are rinsed with a 3% strength aqueous solution of sodium hydroxide before the start of the experiment. The polymerization reactor is then filled with the initial charge.

The initial charge is evacuated prior to the start of the reaction, gassed once with 5 bar of nitrogen, evacuated again and brought to atmospheric pressure with nitrogen. The initial charge is then heated with stirring to a reaction temperature of 75° C. Upon reaching an internal temperature of 70° C., addition 1 is added over the course of two minutes.

Feeds 1 and 2 are metered into the reactor via the dynamic mixer (rotational speed setting 5000 rpm), feed 3 is metered into the reactor via a static mixer which is located in the line section between dynamic mixer and polymerization reactor.

Feed 1 is started directly before feeds 2 and 3. The addition of feed 1 takes place over the course of 1.75 hours, of feed 2 over the course of 1.50 hours, of feed 3 over the course of 3.75 hours.

When feed 3 is complete, the mixture is left to after-polymerize for a further 2 hours with stirring at 75° C. The reaction mixture is then cooled to room temperature and the solids content and also pH are determined.

| | | Measurement value or |
|---|---|---|
| Parameters of the dispersion | Unit | assessment |
| Solids content | (% by wt.) | 31.7 |
| pH | | 9.0 |

The dispersion is then ultrafiltered and the following parameters determined:

| | | Measurement value or |
|---|---|---|
| Parameters of the dispersion | Unit | assessment |
| Solids content | (% by wt.) | 30 |
| Viscosity | (mPas) | 6 |
| pH | | 9.2 |
| LT value | (%) | 85 |
| K value | | 50.5 |
| Average particle size | (nm) | 105 |
| (determined by means of AUC) | | |
| Methanol | (ppm) | 20 |
| Methacrylic acid | (ppm) | 40 |
| N,N-Diethylethanolamine | (ppm) | 210 |
| Storage stability (18 months) | | excellent, no sediment |

### EXAMPLE 5

The procedure is as in example 4, but using potassium persulfate instead of sodium persulfate in the addition 1 and in feed 3.

| | | Measurement value or |
|---|---|---|
| Parameters of the dispersion | Unit | assessment |
| Solids content | (% by wt.) | 30.8 |
| pH | | 9.1 |

The dispersion is then ultrafiltered and the following parameters determined:

| | | Measurement value or |
|---|---|---|
| Parameters of the dispersion | Unit | assessment |
| Solids content | (% by wt.) | 30 |
| Viscosity | (mPas) | 6 |
| pH | | 9.2 |
| LT value | (%) | 89 |
| K value | | 51 |
| Average particle size | (nm) | 110 |
| (determined by means of AUC) | | |
| Methanol | (ppm) | 15 |
| Methacrylic acid | (ppm) | 55 |
| N,N-Diethylethanolamine | (ppm) | 190 |
| Storage stability (after | | excellent, minimal sediment |
| 18 months) | | |

### EXAMPLE 6

The procedure is as in example 4, but using ammonium persulfate instead of sodium persulfate in the addition 1 and in feed 3, and the pH is in each case adjusted to pH 9 using aqueous NaOH.

| | | Measurement value or |
|---|---|---|
| Parameters of the dispersion | Unit | assessment |
| Solids content | (% by wt.) | 30.7 |
| pH | | 8.9 |

The dispersion is then ultrafiltered and the following parameters determined:

| | | Measurement value or |
|---|---|---|
| Parameters of the dispersion | Unit | assessment |
| Solids content | (% by wt.) | 30 |
| Viscosity | (mPas) | 7 |
| pH | | 9.1 |
| LT value | (%) | 85 |
| K value | | 51 |
| Average particle size | (nm) | 115 |
| (determined by means of AUC) | | |
| Methanol | (ppm) | 25 |
| Methacrylic acid | (ppm) | 50 |
| N,N-Diethylethanolamine | (ppm) | 210 |
| Storage stability (after | | excellent, minimal sediment |
| 18 months) | | |

In one variant of the examples given above, it is of course also possible to prepare dispersions with a solids content that is different from 30% by weight. To this end, it is possible, for example, to add demineralized water

a) to the initial charge and/or

b) to feed 1 and/or

c) to feed 3

(with the aim of a lower solids content) or to remove it (with the aim of a higher solids content).

In another embodiment, for example water from the initial charge can be added to feed 1 and/or feed 3, in which case the solids content does not change. The redistribution can, however, also take place by adding the water removed from the initial charge and/or feed 1 and/or feed 3, in its entirety or in part, to a new feed (“feed 4”), feed 4 then being added, continuously or all at once, in parallel to the polymerization, at a later time relative to the polymerization or after the polymerization. This can serve, for example, to adapt the formulation to the tank sizes available, e.g. to avoid overfilling and/or a topping up of feed 1.

It may of course be advantageous to vary the emulsifier distribution disclosed in the examples to the initial charge and feed 1 such that anionic and/or nonionic emulsifier from the initial charge is added to feed 1 (or vice versa). It is of course also possible for anionic and/or nonionic emulsifier from the initial charge and/or feed 1 to be added to an additional feed 4 (cf. above). In all of these measures, the total amount of emulsifier preferably remains constant.

It may of course also be advantageous if feeds 1 and/or feed 2 and/or feed 3 are not metered in at a constant rate, but are introduced at a non-constant rate. For example, the initiator feed can be metered in during the polymerization phase (i.e. during the addition of feed 2) at a higher rate than after feed 2 is complete.

Coating of Administration Forms

### EXAMPLE 7

Coated Ibuprofen Minipellets

Composition of the Pellets

| | Composition per pellet |
|---|---|
| Substance | [%] |
| Ibuprofen | 100 |

Pellet size 100-300 μm

Subcoating:

As subcoating, an aqueous preparation of 5% by weight of Kollicoat® IR (polyvinyl alcohol-polyethylene glycol graft copolymer, PVAI/PEG 75/25, average molecular weight 45 000) and 5% by weight of talc was applied to the ibuprofen minipellets under the spraying conditions of the taste-masking coatings given below. The application amount was 4% by weight, based on the amount of pellets used.

Composition of the Spray Formulation

| | Fraction in the | Fraction in the |
|---|---|---|
| Substance | film [%] | suspension [%] |
| 30% strength aqueous | 65.42 | 43.61 |
| dispersion from example 4 | | |
| Triacetin | 13.08 | 2.62 |
| Talc | 21.5 | 4.3 |
| Water, demineralized | — | 49.47 |
| Total | 100 | 100 |

The plasticizer triacetin was added to the polymer dispersion and left to stir. Talc was slurried in water and homogenized by means of a high-shear mixer. Both preparations were then mixed.

Coating Parameters

Coating was carried out in a “Glatt GPCG 3.1” fluidized-bed granulator from Glatt.

| | |
|---|---|
| Spray nozzle | 1 mm diameter |
| Number of spray nozzles | 1 |
| Charge | 2.5 kg ibuprofen pellets 100-300 μm |
| Method | Bottom spray (Wurster) |
| Spraying pressure | 1.0 bar |
| Incoming air temperature | 58° C. |
| Outgoing air temperature | 47° C. |
| Spraying rate | 10 g/min |
| Spraying time | 4 h |
| Drying | ca. 5 min |
| Application amount/weight | 18% |
| increase | |

Tableting to Give Tablets

All of the ingredients were mixed in a mixer for 10 min and compressed on a rotary press to give tablets.

| | Composition per tablet |
|---|---|
| Substance | [mg] |
| Coated ibuprofen minipellets | 244 |
| Ludiflash ® matrix auxiliary*⁾ | 300 |
| Kollidon ® CL-SF | 20 |
| Sodium stearyl fumarate | 12 |
| Total | 576 |

Format: 11 mm biplane
*⁾Agglomerates of 90% by weight of D-mannitol, 5% by weight of Kollicoat ® SR30 D (polyvinyl acetate/polyvinylpyyrolidone/sodium lauryl sulfate 90/10/0.1), 5% by weight of crospovidone
The particle size of the agglomerates is 150 to 200 μm.

Tablet and Pellet Properties:

Breaking strength: 48N

Release, pellets, paddle, 50 rpm, 37° C. 1000 ml, initial weight 244 mg

| | Acetate buffer pH 4.5 | Phosphate buffer pH 6.8 |
|---|---|---|
| After 45 min | >90% | <10% |

Tablets:

Paddle, 50 rpm, 37° C., 1000 ml

| | Acetate buffer pH 4.5 | Phosphate buffer pH 6.8 |
|---|---|---|
| After 45 min | >90% | <10% |

Disintegration of Tablet:

Disintegration tester Erweka model ZT 74 37° C.

| Acetate buffer pH 4.5 | Phosphate buffer pH 6.8 |
|---|---|
| 21 s | 24 s |

### EXAMPLE 8

Coated Caffeine Pellets

Composition of the Pellets

| | Composition per pellet |
|---|---|
| Substance | [%] |
| Caffeine, fine powder | 20 |
| Avicel PH 101 (MCC) | 38.75 |
| Granulac 230 (Lactose) | 38.75 |
| Kollidon VA 64 | 2.5 |

Preparation by extrusion, pellet size 200-500 μm

Composition of the Spray Formulation

| | Fraction in the | Fraction in the |
|---|---|---|
| Substance | film [%] | suspension [%] |
| 30% strength aqueous | 62.61 | 41.74 |
| dispersion from example 5 | | |
| Acetyl triethyl citrate | 9.39 | 1.88 |
| Iron oxide yellow | 3 | 0.6 |
| Kaolin | 25 | 5 |
| Water, demineralized | — | 50.78 |
| Total | 100 | 100 |

The plasticizer acetyl triethyl citrate was added directly to the cationic polymer dispersion and left to stir. Talc and iron oxide yellow were slurried in water and homogenized by means of an Ultraturrax. Both phases were then mixed by adding the pigment suspension to the polymer dispersion.

Coating Parameters:

Coating was carried out in a “Glatt GPCG 3.1” fluidized-bed granulator from Glatt.

The following conditions were set and/or resulted from the settings:

| Spray nozzle | 1 mm diameter |
|---|---|
| Number of spray nozzles | 1 |
| Charge | 2.5 kg caffeine |
| | pellets 200-500 μm |
| Method | Top spray |
| Spraying pressure | 1.0 bar |
| Incoming air temperature | 62° C. |
| Outgoing air temperature | 44° C. |
| Spraying rate | 15 g/min |
| Spraying time | 125 min |
| Drying | ca. 5 min |
| Application amount/weight increase | 15% |

Tableting to Give Tablets

All of the ingredients were mixed in a mixer for 10 min and compressed on a rotary press to give tablets.

| | Composition per tablet |
|---|---|
| Substance | [mg] |
| Coated caffeine pellets | 115 |
| Ludiflash | 300 |
| Chocolate aroma | 3 |
| Kollidon CL-SF | 10 |
| Sodium stearyl fumarate | 10 |
| Total | 438 |

Format: 10 mm biplane

Tablet and Pellet Properties:

Breaking strength of tablets: 45N

Release, pellets, paddle, 50 rpm, 37° C., 1000 ml, initial weight 115 mg

| | 0.1N HCl | Phosphate buffer pH 6.8 |
|---|---|---|
| After 45 min | >95% | <10% |

Tablets:

Paddle, 50 rpm, 37° C. 1000 ml

| | 0.1N HCl | Phosphate buffer pH 6.8 |
|---|---|---|
| After 45 min | >95% | <10% |

Disintegration of Tablet:

Disintegration tester Erweka model ZT 74, 37° C.

| 0.1N HCl | Phosphate buffer pH 6.8 |
|---|---|
| 22 s | 19 s |

### EXAMPLE 9

### Coated Quinine Sulfate Minipellets

Composition of the Pellets

| | Composition per pellet |
|---|---|
| Substance | [%] |
| Quinine sulfate * 2 H₂O | 10 |
| Avicel PH 101 (MCC) | 46.75 |
| Granulac 230 (Lactose) | 40.75 |
| Kollidon VA 64 | 2.5 |

Pellet size 75-200 μm

Comnosition of the Spray Formulation

| | Fraction in the | Fraction in the |
|---|---|---|
| Substance | film [%] | suspension [%] |
| 30% strength aqueous | 68.26 | 45.51 |
| dispersion from example 6 | | |
| Triacetin | 10.24 | 2.05 |
| Indigotin lake | 1.5 | 0.3 |
| Talc | 20 | 4 |
| Water, demineralized | — | 48.14 |
| Total | 100 | 100 |

The plasticizer triacetin was added directly to the polymer dispersion and left to stir. Talc and indigotin lake were slurried in water and homogenized by means of an Ultraturrax. Both preparations were then mixed by adding the pigment suspension to the polymer dispersion.

Coating Parameters:

Coating was carried out in a “Glatt GPCG 3.1” fluidized-bed granulator from Glatt.

The following conditions were set and/or arose from the settings:

| Spray nozzle | 1 mm diameter |
|---|---|
| Number of spray nozzles | 1 |
| Charge | 1.5 kg quinine sulfate |
| | pellets 75-200 μm |
| Method | Bottom spray (Wurster) |
| Spraying pressure | 1.0 bar |
| Incoming air temperature | 63° C. |
| Outgoing air temperature | 48° C. |
| Spraying rate | 10 g/min |
| Spraying time | 150 min |
| Drying | ca. 5 min |
| Application amount/weight increase | 20% |

Tableting to Give Tablets

All of the ingredients were mixed in a mixer for 10 min and compressed on a rotary press to give tablets.

| | Composition per tablet |
|---|---|
| Substance | [mg] |
| Coated quinine sulfate pellets | 120 |
| Ludiflash | 350 |
| Sodium stearyl fumarate | 10 |
| Total | 480 |

Format: 10 mm biplane

Tablet and Pellet Properties:

Breaking strength of tablets: 43N

Release, pellets, paddle, 50 rpm, 37° C., 1000 ml, initial weight 120 mg

| | 0.1N HCl | Phosphate buffer pH 6.8 |
|---|---|---|
| After 45 min | >95% | <10% |

Tablets:

Paddle, 50 rpm, 37° C. 1000 ml

| | 0.1N HCL | Phosphate buffer pH 6.8 |
|---|---|---|
| After 45 min | >95% | <10% |

Disintegration of Tablet:

Disintegration tester Erweka model ZT, 74, 37° C.

| 0.1N HCl | Phosphate buffer pH 6.8 |
|---|---|
| 24 s | 25 s |

### COMPARATIVE EXAMPLES

Analogously to example 9, the quinine sulfate minipellets were coated with the same application amount of the following products:

Opadry® TM

Eudragit® EPO

Eudragit® RL 30 D

Ethylcellulose

The coated pellets were likewise compressed to give tablets and analyzed, with a test taste of the tablets additionally being carried out. To determine the taste, the pellets or the tablet were gently moved in the mouth using the tongue for 5 min.

Result:

| | Release by | | | | | |
|---|---|---|---|---|---|---|
| | pellets | | Release by tablets | | | |
| | (45 min) | | (45 min) | | | |
| | | Phosphate | | Phosphate | | |
| | 0.1N | buffer | 0.1N | buffer | Taste | |
| | HCl | pH 6.8 | HCl | pH 6.8 | Pellets | Tablets |
| Polymer | >95% | <10% | >95% | <10% | Not bitter, | Not bitter, |
| dispersion | | | | | neutral | slightly |
| according | | | | | | sweet |
| to the | | | | | | |
| invention | | | | | | |
| Opadry | >95% | >95% | >95% | >95% | Very bitter | Very bitter |
| TM | | | | | | |
| Eudragit | >95% | 31 | >95% | 41% | Slightly | Clearly |
| EPO | | | | | bitter | bitter |
| Eudragit | 54% | 44% | 75% | 64% | Slightly | Bitter |
| RL 30 D | | | | | bitter | |
| Ethyl- | 65% | 54% | >80% | 74% | Slightly | Bitter |
| cellulose | | | | | bitter | |

It was found that a bitter taste arose for the products Opadry TM, Eudragit EPO, Eudragit RL 30 D and ethylcellulose particularly in tablet form. This more bitter taste compared to the pellets is attributed to the instability of the coating during tableting. By contrast, no bitter taste was established for the polymer dispersion according to the invention.

1. An orally disintegrating dosage form comprising active ingredients and a coating of polymers, the coating of polymers comprising N,N-diethylaminoethyl methacrylate (DEAEMA) polymerized therein, wherein the active ingredients are embedded into an orally disintegrating matrix and wherein the coating of polymers provides a taste-masking effect.

2. The dosage form according to claim 1, wherein the coating of polymers comprises by weight, based on the total weight of the monomers used for the polymerization:
(a) 43 to 47% of N,N-diethylaminoethyl methacrylate, and
(b) 53 to 57% at least one free-radically polymerizable compound selected from esters of α,β-ethylenically unsaturated mono- and dicarboxylic acids with C₁-C₈ alkanols,
as the only monomers polymerized therein.

3. The dosage form according to claim 1, wherein the matrix is based on sugars and gelatin.

4. The dosage form according to claim 1, wherein the matrix is based on sugars or sugar alcohols or mixtures thereof.

5. The dosage form according to claim 1, wherein the matrix comprises a disintegrant.

6. The dosage form according to claim 5, wherein the disintegrant is selected from the group consisting of crosslinked polyvinylpyrrolidone (crospovidone), croscarmellose, and crosslinked carboxymethylcellulose.

7. The dosage form according to claim 1, wherein the matrix comprises a sparingly water-soluble polymer.

8. The dosage form according to claim 1, wherein the matrix comprises, by weight based on the total weight of the matrix, a co-processed mixture of
a) 60-98% at least one sugar or sugar alcohol or mixtures thereof,
b) 1-25% a disintegrant,
c) 1-15% water-insoluble polymers,
d) 0-15% water-soluble polymers, and
e) 0-15% further auxiliaries,
wherein the sum of components a) to e) is 100% by weight.

9. The dosage form according to claim 1, wherein the coating of polymers comprises by weight, based on the total weight of the monomers used for the polymerization:
(a) 43 to 47% of N,N-diethylaminoethyl methacrylate, and
(b) 53 to 57% at least one free-radically polymerizable compound selected from esters of
α,β-ethylenically unsaturated mono- and dicarboxylic acids with C₁-C₈ alkanols.

10. The dosage form according to claim 9, wherein the coating of polymers further comprises at least one additional monomer (c).

11. The dosage form according to claim 10, wherein the additional monomer (c) is selected from esters of α,β-ethylenically unsaturated mono- and dicarboxylic acids with C₉-C₃₀ alkanols and C₂-C₃₀ alkanediols, amides of α,β-ethylenically unsaturated mono- and dicarboxylic acids with C₂-C₃₀ aminoalcohols which have a primary or secondary amino group, primary amides of α,β-ethylenically unsaturated monocarboxylic acids and N-alkyl and N,N-dialky derivatives thereof, N-vinyllactams, open-chain N-vinylamide compounds, esters of vinyl alcohol and allyl alcohol with C₁-C₃₀ monocarboxylic acids, vinyl ethers, vinyl aromatics, vinyl halides, vinylidene halides, C₂-C₈ monoolefins, unsaturated nitriles, nonaromatic hydrocarbons with at least two conjugated double bonds, and mixtures thereof.

12. The dosage form according to claim 9, wherein the coating of polymers further comprises at least one additional monomer (d).

13. The dosage form according to claim 12, wherein the additional monomer (d) comprises a free-radically polymerizable α,β-ethylenically unsaturated double bond and at least one cationogenic and/or cationic group per molecule polymerized therein, with the proviso that the additional monomer (d) is not N,N-diethylaminoethyl methacrylate.

14. The dosage form according to claim 12, wherein the additional monomer (d) is selected from esters of α,β-ethylenically unsaturated mono- and dicarboxlic acids with amino alcohols, amides of α,β-ethylenically unsaturated mono- and dicarboxlic acids with diamines which have at least one primary or secondary amino group, N,N-diallyalmines and N,N-dially-N-alkylamines and their acid addition salts and quaternization products, vinyl- and allyl-substituted nitrogen heterocycles, and mixtures thereof.

15. The dosage form according to claim 9, further comprising at least one pharmaceutically acceptable auxiliary.

16. The dosage form according to claim 15, wherein the pharmaceutically acceptable auxiliary is selected from aroma substances, taste-improving substances, sweetening agents, glidants, wetting agents, release agents, plasticizers, antiadhesives, stabilizers, pore formers, neutralizing agents, gloss agents, dyes, pigments, disinfectants, preservative, thickeners, or mixtures thereof.

17. The dosage form according to claim 2 wherein the matrix comprises, by weight based on the total weight of the matrix, a co-processed mixture of
a) 60-98% at least one sugar or sugar alcohol or mixtures thereof,
b) 1-25% a disintegrant,
c) 1-15% water-insoluble polymers,
d) 0-15% water-soluble polymers, and
e) 0-15% further auxiliaries,
wherein the sum of components a) to e) is 100% by weight.

18. A method of preparing an orally disintegrating dosage form, the method comprising:
preparing an aqueous polymer dispersion, and
coating the dispersion onto an active ingredient,
wherein the dispersion is obtained by free-radical emulsion polymerization of a monomer mixture comprising N,N-diethylaminoethyl methacrylate, and at least one compound selected from esters of α,β-ethlenically unsaturated mono- and dicarboxylic acids with C₁-C₈ alkanols.

19. The method of claim 18, wherein the dispersion comprises, by weight, based on the total weight of the monomers used for the polymerization:
43 to 47% N,N-diethylaminoethyl methacrylate, and
53 to 57% at least one compound selected from esters of α,β-ethylenically unsaturated mono- and dicarboxylic acids with C₁-C₈ alkanols.

20. The method of claim 18, wherein the aqueous polymer dispersion is in an aqueous medium at a pH of at least 7.
